(19) United States
(12) Patent Application Publication
(10) Pub. No.: US 2007/0082395 A1
(43) Pub. Date: Apr. 12, 2007

(54) Production of normal human osteoclasts

(76) Inventor: Biancamaria Baroli, Cagliari (IT)

Correspondence Address: PEARL COHEN ZEDEK LATZER, LLP, 1500 BROADWAY 12TH FLOOR, NEW YORK, NY, 10036, US

(21) Appl. No.: 11/545,772

(22) Filed: Oct. 11, 2006

Related U.S. Application Data
(60) Provisional application No. 60/724,801, filed on Oct. 11, 2005.

(51) Int. Cl. C12N 5/06; C12N 5/08

(52) U.S. Cl. 435/325; 435/366

(57) ABSTRACT

The present invention provides a method of producing a cell population enriched for osteoclasts in which bone marrow mononuclear cells (BMNC) are cultured in a medium supplemented with growth factors, cytokines, or a combination thereof for at least 7 days to obtain colony-forming units of granulocytes and macrophages (CFU-GM); CFU-GM are isolated from culture by centrifugation; and isolated CFU-GM are cultured for a period of time and under conditions sufficient for CFU-GM to differentiate to osteoclasts. The invention further provides a cell population produced by the methods described.

### CROSS REFERENCE TO RELATED APPLICATIONS

This Application claims the benefit of U.S. Provisional Application Ser. No. 60/724,801, filed Oct. 11, 2005, which is hereby incorporated by reference in its entirety.

### BACKGROUND OF THE INVENTION

Mammalian bone tissue goes through repeated cycles—of bone formation and resorption in a process called bone remodeling. This process serves to repair micro-damage and is essential to maintaining strong bones. In most cases, bones become larger, heavier and denser during childhood and early adulthood, when bone formation proceeds at a higher rate than bone resorption. Bone mass then usually remains stable for a few years during adulthood where equilibrium between bone formation and resorption exists, which is balanced by an interaction between osteoclasts and osteoblasts. Hormones (i.e., cortisol, estrogen, progesterone, parathyroid hormone, calcitonin, insulin) also play an important role in regulating bone remodeling.

Osteoclasts are large, multinucleated giant cells. They are usually extremely rare in bone, although the numbers are increased at sites of active bone turnover. Osteoclasts attach to the bone surface and create a ruffled border, which is comprised of a series of fine finger-like cytoplasmic projection of the plasma membrane adjacent to the bone. Resorption and degradation of mineralized bone matrix occur beneath the ruffled border due to the release of proteolytic enzyme and hydrogen ions across the ruffled border into the sealing zone. Osteoclasts derive from bone marrow mononuclear cells. The differentiation, recruitment and inhibition of osteoclasts are controlled by numerous hormonal and growth factors.

To study the cellular pathogenesis and discover therapeutic agents for metabolic bone diseases, methods for the isolation and characterization of osteoclasts and osteoblasts are required.

Osteoclast formation and activity under physiological conditions are controlled by interactions with osteoblasts. Thus, osteoclasts can be generated from hematopoietic cells from spleen or bone marrow in co-cultures of osteoblasts or stromal cells. Also, culture systems using specific growth factors such as macrophage-colony stimulating factor (M-CSF) and/or receptor activator of NF-κB ligand (RANKL) have also been used to generate osteoclasts.

However, these protocols are not robust enough to ensure production of highly active cells in a relatively stable fashion. Protocols which produce high levels of osteoclasts with a minimal amount of steps, (separating cell populations, isolating non-adhesive cells) are needed to study osteoclast physiology, osteoclast communication with other cell types within the bone and bone marrow, in vitro models of osteoclast-related pathologies and treatments, osteoclast-osteoblast equilibrium, and for tissue engineering applications.

### SUMMARY OF THE INVENTION

The present invention provides a method of producing a cell population enriched for osteoclasts in which bone marrow mononuclear cells (BMNC) are cultured in a medium supplemented with growth factors, cytokines, or a combination thereof for at least 7 days to obtain colony-forming units of granulocytes and macrophages (CFU-GM); CFU-GM are isolated from culture by centrifugation; and isolated CFU-GM are cultured for a period of time and under conditions sufficient for CFU-GM to differentiate to osteoclasts. The invention further provides a cell population enriched for osteoclasts produced by the methods described.

### DETAILED DESCRIPTION OF THE PRESENT INVENTION

The present invention provides a method of producing a cell population enriched for osteoclasts in which bone marrow mononuclear cells (BMNC) are cultured in a medium supplemented with growth factors, cytokines, or a combination thereof for at least 15 days to obtain colony-forming units of granulocytes and macrophages (CFU-GM); CFU-GM are isolated from culture by centrifugation; and isolated CFU-GM are cultured for a period of time and under conditions sufficient for said CFU-GM to differentiate to osteoclasts.

In one embodiment, this method comprises culturing BMNC in a medium supplemented with growth factors, cytokines, or a combination thereof for at least 7 days to obtain colony-forming units of granulocytes and macrophages (CFU-GM). In one embodiment, hematopoietic stem cells, hematopoietic progenitors, primitive hematopoietic progenitors, primitive Lin⁻CD34⁻ cells are cultured according to the methods of the present invention to obtain CFU-GM. In another embodiment, thymus mononuclear cells, spleen mononuclear cells, peripheral blood mononuclear cells, G-CSF mobilized mononuclear cells, cord blood mononuclear cells, bone marrow CD34+ cells, G-CSF mobilized CD34+ cells, cord blood CD34+ cells, fetal liver CD34+ cells, bone marrow AC133+ cells, G-CSF mobilized AC133+ cells, cord blood AC133+ cells, fetal liver AC133+ cells, myeloid CD33+ cells, leukemic or promyelocytic cell lines, joint fluid of rheumatoid arthritis patients, or human giant bone cell tumors are cultured to obtain CFU-GM. In one embodiment, cells cultured to obtain CFU-GM may be early osteoclast precursor cells or committed osteoclast precursor cells.

In one embodiment, this method comprises culturing BMNC in a medium supplemented with growth factors, cytokines, or a combination thereof for at least 7, or at least 10, or at least 15, or at least 20, or at least 25, or at least 30, or at least 35 days to obtain CFU-GM. According to this aspect of the invention, and in one embodiment, BMNC are isolated according to the following protocol: Bone marrow is obtained from the iliac crest of human volunteers under anesthesia and collected into syringes containing α-MEM with 100 U/ml of heparin or an alternative anti-coagulant and 5% serum. Mononuclear cells are collected after Ficoll-Hypaque gradient centrifugation, ammonium chloride precipitation, or any method known in the art to separate mononuclear cells. In one embodiment, cells are layered over Ficoll-Paque (Amersham Pharmacia), centrifuged at 400 g for 30 minutes at room temperature, then collected.

In one embodiment, this method comprises culturing BMNC or other cells as described hereinabove in a medium supplemented with growth factors, cytokines, or a combination thereof for at least 7 days to obtain CFU-GM. According to this aspect of the invention, and in one embodiment, BMNC or other cells as described hereinabove are isolated from tissue using any method known in the art, which, in one embodiment may be positive immuno-magnetic selection (CD34+ or CD133+ Progenitor Cell Isolation Kit from Miltenyi Biotec, or CD34+ or CD133+ progenitor Cell Positive Selection or Bone Marrow Progenitor Pre-enrichment Kits from Stem Cell Technologies.) In another embodiment, the antibodies used for these procedures may be conjugated to magnetic beads and immunogenic procedures may be utilized to recover the desired cell type. In another embodiment, bone marrow mononuclear cells or other cells as described hereinabove can be recovered from human tissue by depletion, i.e., by selecting out cells which express markers found on other undesired cells.

In one embodiment, the method comprises culturing peripheral blood mononuclear cells in a medium supplemented with growth factors, cytokines, or a combination thereof for at least 7 days to obtain CFU-GM. In one embodiment, the method comprises culturing peripheral blood mononuclear cells in a medium supplemented with growth factors, cytokines, or a combination thereof for at least 5 days, or in another embodiment, at least 7 days, to obtain CFU-GM. In some embodiments of this invention, the CFU-GM are already visible after 7 days. At the lowest dilution used in experiments conducted herein, i.e. with 1,560 cell/1.1 ml from 1 to 6 colonies was found.

According to this aspect of the invention, and in one embodiment, peripheral blood mononuclear cells are isolated from whole blood or buffy coat and layered over Ficoll-Paque following 1:2 dilution in PBS. Leukocytes are collected and then washed.

In one embodiment, this method comprises culturing BMNC in a medium supplemented with growth factors, cytokines, or a combination thereof for at least 3, or in another embodiment, at least 5, or in another embodiment, at least 7, or in another embodiment, at least 10, or in another embodiment, at least 15 days to obtain CFU-GM. According to this aspect of the invention, and in one embodiment, BMNC or other pluripotent cells may be derived from any species, which are able to form osteoclasts. In one embodiment, cells are derived from mammal tissue. In another embodiment, cells are derived from bovine, rodent, or primate tissue. In another embodiment, cells are derived from mouse, rat, or monkey tissue. In another embodiment, cells are derived from human tissue. In another embodiment, cells are derived from other species whose osteoclasts share biochemical characteristics with human osteoclasts.

According to the methods of the present invention, a medium supplemented with growth factors, cytokines, or a combination thereof is used to culture BMNC for at least 3, or in another embodiment, at least 5, or in another embodiment, at least 7, or in another embodiment, at least 10, or in another embodiment, at least 15 days to obtain CFU-GM. In one embodiment, the medium for use in the present invention is viscous. In another embodiment, it is non-viscous. In another embodiment, it may be any medium known in the art that is used for cell culture. In another embodiment, the medium may comprise alpha-minimum essential medium (MEM) or Iscove's modified dulbecco's medium (IMDM), while in another embodiment, it may comprise a commercially sold medium, such as Mesenchymal Stem Cell Bulletkit medium (MSCGM medium; Poietics, BioWittaker, Walkersville, Md.).

In one embodiment, the medium used to culture BMNC is viscous. According to this aspect of the invention and in one embodiment, the medium may comprise, in one embodiment, methylcellulose. In another embodiment, viscous medium may comprise plasma gel or fibrin clots. In another embodiment, a medium for culturing BMNC to obtain CFU-GM according to the methods of the present invention may comprise agar, in another embodiment, soft agar.

In another embodiment, a medium for culturing BMNC to obtain CFU-GM according to the methods of the present invention may comprise fetal bovine serum (FBS), fetal calf serum (FCS), bovine serum albumin (BSA), mercaptoethanol, L-glutamine, or a combination thereof. In another embodiment, the medium for culturing BMNC may further comprise estrogens, biphosphonates, or a combination thereof. In another embodiment, a medium may further comprise antibiotics.

In one embodiment, the growth factor, cytokine or a combination thereof used to supplement medium for culturing BMNC to obtain CFU-GM according to the methods of the present invention is granulocyte macrophage colony stimulating factor (GM-CSF), IL-3, stem cell factor, or a combination thereof. In one embodiment, the medium comprises GM-CSF. In one embodiment, GM-CSF increases the osteoclast progenitor population size. In one embodiment, the medium comprises IL-3. In one embodiment, the medium comprises stem cell factor. In another embodiment, the growth factor, cytokine or a combination thereof used to supplement medium is receptor activator of NF-κB ligand (RANKL), macrophage colony stimulating factor (M-CSF), transforming growth factor (TGF)-α, TGF-β, Interleukin (IL)-1, IL-6, IL-7, IL-17, tumor necrosis factor (TNF)-α, parathyroid hormone (PTH), 1,25-dihydroxy Vitamin D3, calcitonin, dexamethasone, eotaxin, eotaxin-2, eotaxin-3, or any combination thereof.

In one embodiment, BMNC is cultured in a medium supplemented with growth factors, cytokines, or a combination thereof for at least 3 days, or in another embodiment, at least 5 days, or in another embodiment, at least 7 days, or in another embodiment, at least 10 days, or in another embodiment, at least 15 days, or in another embodiment, at least 20 days, or in another embodiment, at least 25 days, or in another embodiment, at least 30 days, or in another embodiment, at least 35 days, to obtain CFU-GM. According to this aspect of the invention and in one embodiment, BMNC may be co-cultured with other cell types. In one embodiment, BMNC may be co-cultured with mesenchymal, osteoblast or stromal cells.

In one embodiment, BMNC cells are cultured in medium supplemented with growth factors, cytokines, or a combination thereof for at least 7 days, or in another embodiment, at least 10 days, or in another embodiment, at least 15 days to obtain CFU-GM. In another embodiment, they are cultured for 15-35 days. In another embodiment, they are cultured for 25 days.

In one embodiment, BMNC cells are cultured at low concentration to obtain CFU-GM. In one embodiment, the BMNC are cultured at a concentration of at least 5,000, or in another embodiment, at least 10,000 cells in 1.1 ml of the culture media. Various concentrations, ranging from 6,250 to 1,560 in 1.1 ml were employed herein, with a few recoverable GM-CFU colonies resulting. In some embodiments 12,500 cells/1.1 ml of culture yield greater numbers of GM-CFU colonies. The following cell quantities were evaluated: 50,000; 25,000; 12,500; 6,250; 3,120; 1,560, with the results as indicated, wherein 12,500 or greater produced more recoverable GM-CFU colonies.

Cells seeded at 12,500 or greater produced both greater numbers of colonies, as well as cells per colony. Thus, in some embodiments, the methods of this invention provide for greater numbers of GM-CFU colonies, or greater numbers of cells per colony, or a combination thereof, which in turn results in greater osteoclast progenitor production, in some embodiments, and ultimately, in some embodiments, greater osteoclast production.

In some embodiments, GM-CSF overnight treatment was stimulatory, in terms of promoting GM-CFU colonly/cell per colony expansion/production/osteoclast progenitor production/osteoclast production, etc., only at high BMNC occupany, for example, as demonstrated herein with at least 50,000 BMNC were seeded in 35 mm Petri dishes. Such high seeding numbers of BMNC in cultures, as herein described, results in greater recovery, sooner of GM-CFU, in some embodiments.

According to this aspect of the invention and in one embodiment, BMNC cultured at a concentration of 50,000-1,000,000 cells/ml produce a population of osteoclast progenitor cells, which in turn ultimately yielded a population of mature, differentiated osteoclasts. In another embodiment, BMNC are cultured at 50,000-75,000 cells/ml. In another embodiment, BMNC are cultured at a concentration of approximately 100,000 cells/ml. In another embodiment, BMNC are cultured at a concentration of 5,000 cell/ml. In another embodiment, BMNC are cultured at a concentration of 10,000 cell/ml. In another embodiment, BMNC are cultured at a concentration of 150,000 cell/ml. In another embodiment, BMNC are cultured at a concentration of 200,000 cell/ml. In another embodiment, BMNC are cultured at a concentration of 250,000 cell/ml. In another embodiment, BMNC are cultured at a concentration of 300,000 cell/ml. In another embodiment, BMNC are cultured at a concentration of 350,000 cell/ml. In another embodiment, BMNC are cultured at a concentration of 400,000 cell/ml. In another embodiment, BMNC are cultured at a concentration of 450,000 cell/ml. In another embodiment, BMNC are cultured at a concentration of 500,000 cell/ml. In another embodiment, BMNC are cultured at a concentration of 650,000 cell/ml. In another embodiment, BMNC are cultured at a concentration of 800,000 cell/ml.

In one embodiment, CFU-GM in supplemented medium may be identified during or after 15 days of incubation. According to this aspect of the invention and in one embodiment, CFU-GM may be identified using any method known in the art. In another embodiment, CFU-GM are identified using a microscope, in another embodiment, an inverted microscope, in another embodiment, using the naked eye. In one embodiment, CFU-GM are detected by the specific morphology of the cells. In another embodiment, CFU-GM are identified with May-Grtmwald Giemsa stain, while in another embodiment, with anti-CD 14 and anti-CD66b. In another embodiment, CFU-GM are identified by CD13, CD33, CD34, human lymphocyte antigen D-related (HLA-DR) markers, or a combination thereof.

In one embodiment, CFU-GM are isolated from supplemented medium after at least 5, or in another embodiment, at least 7, or in another embodiment, at least 10, or in another embodiment, at least 15 days using any means known in the art. According to this aspect and in one embodiment, CFU-GM may be isolated from culture by centrifugation. According to this aspect and in one embodiment, CFU-GM may be isolated from culture by centrifugation at a speed, for a period of time, and with a number of repetitions that can be determined by one skilled in the art to isolate CFU-GM. In one embodiment, CFU-GM may be isolated by centrifugation at 1200 rpm, or in another embodiment, at 300 g for 15 minutes. In another embodiment, CFU-GM may be isolated by centrifugation for 10 minutes. In another embodiment, CFU-GM may be isolated by using a centrifugation protocol that is repeated multiple times, which in one embodiment, may be 2 times, while in another embodiment, 3 times. In another embodiment, CFU-GM may be isolated by centrifugation at 1200 rpm for 10 minutes for 2-3 repetitions. In another embodiment, CFU-GM may be isolated by centrifugation at 300 g for 15 minutes.

In another embodiment, basal medium is used to wash the pellet between centrifugation cycles, which basal medium is, in one embodiment, IMDM. In another embodiment, wash medium is aspirated and a pellet is recovered after centrifugation.

In another embodiment, CFU-GM produced from culturing BMNC for at least 7, or in another embodiment, at least 10, or in another embodiment, at least 15 days in supplemented medium may be isolated from culture manually, which, in one embodiment, comprises picking colony as is known to one skilled in the art. In one embodiment, CFU-GM are picked using a finely drawn pipette. In another embodiment, CFU-GM are isolated using, in some embodiments, any separation means known in the art, for example, magento- or immuno- or immunomagneto-separation. In some embodiments, such separation methods may comprise use of a column or magnetic beads.

According to the methods of the present invention, CFU-GM are cultured for a period of time and under conditions sufficient for CFU-GM to differentiate to osteoclasts. In one embodiment, the medium for culturing CFU-GM to produce osteoclasts is any medium described hereinabove. In one embodiment, the CFU-GM culture medium is non-viscous. In one embodiment, the CFU-GM culture medium is IMDM comprising fetal bovine serum. In one embodiment, the medium for culturing CFU-GM to produce osteoclasts comprises growth factors, cytokines, or a combination thereof, as known in the art or as described hereinabove. In another embodiment, the CFU-GM culture medium comprises M-CSF and RANKL. In one embodiment, M-CSF and RANKL promote osteoclastogenesis.

According to the methods of the present invention, CFU-GM are cultured on an appropriate surface that is known in the art for a period of time and under conditions sufficient for CFU-GM to differentiate to osteoclasts. According to this aspect and in one embodiment, CFU-GM are cultured on 24-well plates, while in another embodiment, CFU-GM are cultured on 4-well chamber slides, while in another embodiment, they are incubated on slides.

In one embodiment, the methods of this invention include, inter alia, producing a cell population enriched for osteoclasts, via culturing bone marrow mononuclear cells (BMNC) in a medium supplemented with growth factors, cytokines, or a combination thereof, for at least 7 days to obtain colony-forming units of granulocytes and macrophages (CFU-GM), where the BMNC are not previously separated into adherent versus nonadherent populations prior to culturing them with the supplemented medium. CFU-GM are then isolated by centrifugation, and not manual isolation, in one embodiment. In some embodiments, lack of the BMNC separation step, or manual selection of CFU-GM is an enormous time-saving step, and in some embodiments, enhances the likelihood of obtaining sterile, robust osteoclast progenitor populations. The CFU-GM are then cultured for a period of time and under conditions sufficient for the CFU-GM to differentiate to osteoclasts.

In one embodiment, osteoclasts produced via the methods of the present invention are at any one of various stages of differentiation. In one embodiment, osteoclasts produced by the methods of the present invention express various osteoclast markers. In one embodiment, osteoclasts express at least one osteoclast-specific marker. In one embodiment, the cells obtained by the methods of this invention function as mature, differentiated osteoclasts, which are capable of bone resorption. In one embodiment, mature, differentiated osteoclasts may be identified by one or more of the following characteristics: multiple nucleii, resistance to tartaric acid, formation of an actin ring structure and polar cell body during resorption, contraction and/or immobilization in response to calcitonin, formation of resorption lacunae, ruffled border, or a combination thereof. In another embodiment, osteoclasts may be identified by the presence of osteoclast markers. In one embodiment, osteoclast markers comprise tartrate-resistance acid phosphatase (TRAP), calcitonin receptor, receptor activator of NF-κB (RANK; RANKL receptor), c-fms (M-CSF receptor), cathepsin K, c-src, fosL1and vitronectin receptor (αVβ3 integrin), osteopontin (OP), carbonic anhydrase II (CAII), MMP-9, or a combination thereof. In another embodiment, mature, differentiated osteoclasts may be identified by expression of Kn22⁺, CD11b, or a combination thereof.

In one embodiment, osteoclasts obtained using the methods of the present invention are capable of bone resorption. In another embodiment, osteoclasts produced by the methods of this invention may be distinguished from non-osteoclasts by their ability to resorb bone. According to this aspect and in one embodiment, bone resorption refers to bone dissolution, while in another embodiment, it refers to bone loss. In one embodiment, bone resorption may be evaluated by the ability of osteoclasts to form resorption pits on bone or dentine slices, which in one embodiment, may be bovine cortical bone or ivory dentin slices. In another embodiment, bone resorption is evaluated by the ability of osteoclasts to form resorption pits on synthetic materials, which in one embodiment are polymeric scaffolds. In another embodiment, the ability of osteoclasts to form resorption pits on synthetic materials coated with calcium phosphate, or in another embodiment, with synthetic hydroxyapatite, is evaluated.

In another embodiment, osteoclasts produced by the methods of the present invention are not fully mature. In one embodiment, cells produced by the methods of the present invention are early osteoclast precursors, which in one embodiment, may be identified by their expression of MMP-9, TRAP, or a combination thereof and, in another embodiment, by their expression of Kn22⁺ CD11b⁺, and My11⁺. In another embodiment, cells produced by the methods of the present invention are committed osteoclast precursors, which in one embodiment, may be identified by their expression of MMP-9, TRAP, and vitronectin receptor, and, in another embodiment, by their expression of CD45A, Kn22⁺, CD11b, vitronectin receptor, and calcitonin receptor. In another embodiment, cells produced by the methods of the present invention are immature osteoclasts, which in one embodiment, may be identified by their expression of CD45A, Kn22⁺, CD11b, vitronectin receptor, and calcitonin receptor.

In one embodiment, osteoclasts produced by the methods of the present invention are activated (which in one embodiment refers to the ability to resorb), while in other embodiments, they are not activated. In one embodiment, methods of the present invention comprise an additional step wherein osteoclasts are activated for resorption. In one embodiment, this step comprises adding to the osteoclast culture medium: IL-1α, IL-6, calcitonin, TGF-β, M-CSF, or a combination thereof.

In one embodiment, this invention provides a method of producing a cell population enriched for osteoclasts. According to this aspect and in one embodiment, “enriched” refers to a cell population in which 65-99.9% of the cells display osteoclast markers and/or functions. In another embodiment, at least 90-100% display osteoclast markers and/or functions. In another embodiment, at least 95-100% display osteoclast markers and/or functions. In another embodiment, at least 75% display osteoclast markers and/or functions. In another embodiment, at least 85% display osteoclast markers and/or functions. In another embodiment, at least 90% display osteoclast markers and/or functions. In another embodiment, at least 95% display osteoclast markers and/or functions. In another embodiment, at least 99% display osteoclast markers and/or functions.

In one embodiment, the method of the present invention which produce an osteoclast-enriched cell population additionally comprises a step in which BMNC are pre-incubated in non-viscous medium for 8-24 hours prior to the step of incubating BMNC for greater than 7, or in another embodiment 10, or in another embodiment 15 days to produce CFU-GM. In one embodiment, the method of producing an osteoclast-enriched cell population does not comprise a pre-incubation step. In one embodiment, the pre-incubation medium is any of the media described hereinabove and comprises growth factors, cytokines, or a combination thereof as described hereinabove. In another embodiment, the pre-incubation medium comprises GM-CSF, while in another embodiment, it does not comprise GM-CSF. In one embodiment, the incubation period is overnight, in another embodiment, 8-20 hours, in another embodiment 15 hours. In one embodiment, non-adherent cells are separated from adherent cells after the pre-incubation step, while in one embodiment, they are not separated after the pre-incubation step. In another embodiment, the medium is centrifuged after pre-incubation to separate non-adherent from adherent cells.

According to this aspect and in one embodiment, the method of the present invention produces a cell population enriched for osteoclasts. According to this aspect and in one embodiment, “enriched” refers to cell population in which 65-99.9% of the cells display osteoclast markers and/or functions. In another embodiment, 85-90% display osteoclast markers and/or functions. In another embodiment, at least 75% display osteoclast markers and/or functions. In another embodiment, at least 85% display osteoclast markers and/or functions. In another embodiment, at least 90% display osteoclast markers and/or functions.

In another embodiment, the present invention provides a kit for producing an osteoclast-enriched cell population comprising: BMNC, viscous medium supplemented with growth factors, cytokines, or a combination thereof for culturing BMNC to obtain colony-forming units of granulocytes and macrophages (CFU-GM); and non-viscous medium supplemented with growth factors, cytokines, or a combination thereof for culturing CFU-GM. In another embodiment, the kit further comprises an additional non-viscous medium for pre-incubation of BMNC. In another embodiment, the kit further comprises wash medium, which in one embodiment, is a basal medium, which is, in one embodiment, IMDM. In another embodiment, the kit comprises thymus mononuclear cells, spleen mononuclear cells, peripheral blood mononuclear cells, G-CSF mobilized mononuclear cells, cord blood mononuclear cells, bone marrow CD34+ cells, G-CSF mobilized CD34+ cells, cord blood CD34+ cells, fetal liver CD34+ cells, bone marrow AC133+ cells, G-CSF mobilized AC133+ cells, cord blood AC133+ cells, fetal liver AC133+ cells, myeloid CD33+ cells, hematopoietic stem cells, hematopoietic progenitors, primitive hematopoietic progenitors, primitive Lin⁻CD34⁻ cells, leukemic or promyelocytic cell lines, human giant bone cell tumors, etc in place of or in addition to BMNC. In another embodiment, the kit comprises components used to extract BMNC from bone marrow, peripheral blood mononuclear cells, etc as described hereinabove.

In one embodiment, the kit will comprise, in a suitable container means, the cells and reagents useful for the methods of the present invention, and any additional agents that may be used in accordance with the present invention. The kits may comprise suitably aliquoted compositions of the present invention. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. A kit of the present invention also will typically include a means for containing reagent containers in close confinement for commercial sale. Such containers may include injection or blow-molded plastic containers into which the desired vials are retained. When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

In one embodiment, the present invention provides a population of cell enriched in osteoclasts that is produced by the process described hereinabove.

In one embodiment, the methods of the invention also provide a population of cells originating from human or other donor tissue that are grown in ex vivo culture. In another embodiment, the methods of the invention are provide a cell population for use in autologous cell transfer, in which bone marrow cells are removed from the patient, grown or maintained in an ex vivo culture, and then transplanted back into the patient. Accordingly, the methods of the present invention may be employed as a means for increasing the levels of osteoclasts in bone marrow using ex vivo cell culture medium. According to these embodiments of the present invention, the method would comprise an additional step of isolating bone marrow mononuclear cells or other precursor cells that may potentially differentiate into osteoclasts from donor tissue. In one embodiment, donor tissue may be bone marrow, in another embodiment, it may be peripheral blood, in another embodiment, it may be cord blood.

In one embodiment, the cells may be genetically manipulated in ex vivo culture. In one embodiment, genetic manipulation may entail introducing foreign genes into osteoclasts or osteoclast progenitors. According to this embodiment, cells may be genetically manipulated at any stage of differentiation, including at the CFU-GM stage or at the osteoclast stage. In another embodiment, expression vectors comprising foreign genes may comprise one or more selectable marker genes to provide a phenotypic trait for selection of transformed cells such as dihydrofolate reductase or neomycin resistance. In one embodiment, genes may be introduced into cells, which are then returned to the autologous donor or an allogeneic recipient where the expression of the gene will have a therapeutic effect. In one embodiment, osteoclasts may be genetically engineered to have reduced activity in vivo. According to this embodiment, genes that play a role in the regulation of osteoporosis, in areas such as serum calcium responsiveness, estrogen secretion and bone resorption, may be introduced into osteoclasts or osteoclast progenitors.

In one embodiment, methods of the present invention may be used to produce cell populations for research purposes, or in another embodiment, for the purpose of treatment or prevention of a disease, condition, or disorder. In one embodiment, the cell population produced may be useful for understanding osteoclast physiology, osteoclast communication with other cells types within the bone or bone marrow, osteoclast pathologies, osteoclast-osteoblast equilibrium, or a combination thereof. In another embodiment, the cell population produced may be useful for developing drug or bioactive molecules that may be useful in treating or preventing osteoclast-related pathologies.

In another embodiment, the cell population may be used for tissue engineering applications, which in one embodiment, may comprise allowing osteoclasts to resorb a polymeric scaffold for the engineered regeneration of bone tissue. In one embodiment, osteoclast resorption takes place in the presence of osteoblasts, which may then construct additional bone on the polymeric scaffold. In another embodiment, osteoclasts may be encapsulated inside a membrane, which, in one embodiment, is permeable to nutrients and factors produced by osteoclasts, which, in one embodiment, allows the nutrients and factors to leave the encapsulated membrane. According to this aspect and in one embodiment, osteoclasts are subjected to stress to increase production of osteoclast nutrients and factors, which, in one embodiment, increases the number of osteoblasts in the surrounding environment.

In one embodiment, the cell population produced may be useful for treating lesion calcifications, which in one embodiment, may occur in the lung, brain, cranium, coronary artery, aortic, heart, tooth, or other organs as are known in the art. In another embodiment, the cell population produced may be useful for treating dystrophic calcification, which in one embodiment may results from tissue damage, which in one embodiment, is caused by implantation of at least one medical device. In another embodiment, the cell population produced may be useful for treating pleural disease, which in one embodiment, is related to asbestos exposure. In another embodiment, the cell population produced may be useful for treating sarcoidosis.

### EXAMPLES

### Example 1

### Differentiation of Osteoclasts from BMNC

### Materials & Methods

Cryopreserved Bone Marrow Mononuclear Cells (BMNC; Lot # 2F0433) and Mesenchymal Stem Cell Bulletkit medium (MSCGM) were purchased from Poietics (BioWitthaker Inc., Walkersville, Md., USA); Fetal Bovine Serum (FBS; Lot # 1086744; cat # 16000-044), α-MEM (cat. # 12571-055), and cell dissociation buffer were purchased from Gibco™ (Invitrogen corporation, Carlsbad Calif., USA); Iscove's modified dulbecco's medium with 2% fetal bovine serum (2% IMDM), and a complete methylcellulose medium (MethoCult™GF H4534; Lot# 2C144185) were supplied by StemCell Technologies Inc. (Vancouver BC, Canada); Dimethyl sulfoxide (DMSO) and OmniSolv® Methanol (MeOH) were from EM Science (Gibbstown N.J., USA). Recombinant human soluble rank ligand (rh-sRANKL; Lot # 0402142/F062a), recombinant human macrophage colony stimulating factor (rh-M-CSF; Lot # 04130/F141 and 02185/E142a), and recombinant human granulocyte macrophage colony stimulating factor (rh-GM-CSF; Lot# 04130/F141) were obtained from Research Diagnostic Inc. (Flanders N.J., usa). The following compounds were purchased from Sigma-Aldrich (St Louis Mo., USA): dexamethasone, Trypan Blue solution 0.4%, acetone, and Eosin Y (Aldrich), Triton®-X100 (Sigma Ultra), acid phosphatase leukocyte kit (TRAP) and formalin solution neutral buffered (4% form-PBS; formaldehyde 4% w/v) (Sigma Diagnostic). A phosphate buffered saline solution (PBS) was ordered from Roche Diagnostic Corporation (Indianapolis Ind., USA). Fluorescent stains propidiuni iodide (PI; 1.0 mg/mL water; lot# 45A3); Alexa Fluor® 488 phalloidin (Ph; lot# 41B1); YO-PRO®-1 iodide (Yo-pro; 1 mM in DMSO; Lot#2162-9); Hoechst 33342 dye (H; trihydrochloride, trihydrate) and the mounting media SlowFade® Light Antifade Kit were supplied by Molecular Probes Inc. (Eugene Oreg., USA). All products were stored and processed according to supplier instructions.

### Cell Storage and Resuspension

BMNC were stored in liquid nitrogen or immediately prepared for further experiments. Cryovials (25,000,000 cell/ml) were quickly thawed in a 37° C. water bath for 1 minute, wiped with 70% ethanol and transferred to a sterile hood (model VBM-400; The Baker Co., Sanford, Me., USA), where cells were resuspended with an up-and-down pipetting motion.

### Overnight Pretreatment

Three protocols of overnight pre-treatment of BMNC were compared. Group A received no overnight pretreatment, Group B was pre-treated with MSCGM medium, and Group C was pre-treated with filtered (0.22 μm, # 430767, Corning Inc. Life Sciences, Acton Mass., USA) α-MEM containing 5% FBS and rh-GM-CSF (280 μg/mL). For Groups B and C, 300 μL of thawed BMNC were aseptically transferred to a 175T flask (vented cap; Corning Inc. Life Sciences, Acton Mass., USA) containing 35 mL of pre-treatment medium that had been pre-equilibrated at 37° C. in a humidified atmosphere of 5% CO₂ (model 3158; Forma Scientific, Marietta Ohio, USA) for at least 30 minutes. Flasks were then gently rocked to evenly distribute the cells and incubated overnight (15 hours). Subsequently, medium was aseptically aspirated from each flask in a sterile hood and transferred into two 15 mL falcon tubes. Flasks were gently washed twice with 5 mL of equilibrated overnight medium, which was then pooled to the falcon tubes. Non-adherent BMNC were pelleted down at 1000 rpm for 5 min (Labofuge 400, Heraeus Instruments Inc., Newtown Conn., USA), unified and then resuspended in 1 mL of equilibrated 2% IMDM medium. Cell viability was assessed with the trypan blue method.

### Cell Culture

BMNC from Groups A, B, and C were cultured on a methylcellulose-based medium (MethoCult™GF H4534) to trigger and favor their differentiation to mature colony-forming units belonging to the lineage of granulocytes and macrophages (CFU-GM). 3 mL pre-aliquoted MethoCult™GF tubes were thawed at room temperature in a sterile hood for 4 hours. 0.3 mL of BMNC were then aseptically introduced in each tube, and immediately after, mixtures were vortexed vigorously to evenly distribute cells within the viscous medium and then allowed to stand to allow air bubbles to dissipate. Each tube's content was aspirated using a 3 mL new, sterile syringe fitted with a 16-gauge blunt end needle (StemCell Technologies Inc., Vancouver, BC, Canada), and plated in 1.1 mL volume in one of two 35 mm petri dish, which were placed with a third 35 mm petri dish in a 100 mm petri dish. To maintain a constant level of humidity in each 100 mm petri dish, the third 35 mm petri dish was filled with 5 mL Milli-Q sterile water (0.22 μm Stericup™; Millipore, Bedford Mass., USA) and left uncovered. Cells were then incubated undisturbed for 2-3 weeks. BMNC were plated at a final concentration of 8E5 (Group A1), 2E5 (Group A2), 1E5 (Group B), and 2E5 (Group C) cells per 1.1 mL of the final plating mixture.

### Examination

After 10 and 25 days of incubation, dishes were placed one at a time inside a 60 mm gridded dish to examine colonies using an inverted microscope. Colonies were counted and then imaged (Zeiss Axiovert 200; Carl Zeiss Microimaging Inc., Thornwood, N.Y., USA) prior to further incubation.

### Isolation

After 26 days of incubation, cells were isolated according to the following procedure. The 35 mm petri dishes were incubated at 4° C. for 1-2 hours, and then returned to the sterile hood. 35 mm petri dishes were then washed three times with 0.8 mL of equilibrated 2% IMDM using an up-and-down pipetting motion, and the cell-containing mixtures pooled in a 50 mL Falcon tube, to which a further 10 mL of 2% IMDM were added. Mixtures were then centrifuged at 1200 rpm for 10 minutes, two or three times, until a well-defined cell-pellet was formed. After each centrifuge cycle, the supernatant was aspirated leaving 5 mL of mixture in the tube. Cells were resuspended in 10 mL of equilibrated 2% IMDM and re-centrifuged as described above. After the final cycle, supernatant was aspirated, and cells were resuspended in 1 mL of equilibrated 2% IMDM, counted (trypan blue) and diluted to the concentration required for further applications

### Freezing

Mature colony cells were frozen in IMDM medium comprising 30% FBS, supplemented with 10% of DMSO at −80° C. (Nalgene™ Cryo 1° C. freezing container; Nalge Nunc International, Naperville, Ill., USA) for 16 hours, and then stored under liquid nitrogen.

### Osteoclast Formation

Colony cells were seeded at a concentration of approximately 62,000 cells per cm² on either 24 well plates (Costar® 3524; Corning Inc., Life Sciences, Acton Mass., USA) or 4-well chamber slides (Lab-Tek® II Chamber Slides™ System; Nalge Nunc International, Naperville, Ill., USA) filled with 1 or 2 mL of equilibrated osteoclast formation medium (OC-M). The OC-M was composed of IMDM containing 10% FBS, rh-sRANKL (100 ng/mL) and rh-GM-CSF (25 ng/mL). Medium was changed every other day. Cells were cultured for up to 16 days, and then fixed, stained, and imaged.

### TRAP (Tartrate-Resistant Acid Phosphatase) Stain

Cells were removed from the incubator, medium aspirated completely, and cells washed twice with sterile (0.22 μm Stericup™; Millipore, Bedford Mass., USA) PBS. Cells were then fixed and stained according to Sigma procedure # 386 except that slides were incubated for 30 minutes. After staining, slides were mounted with an antifade reagent in glycerol buffer (SlowFade® Light) and sealed with acrylic glue.

### Hematoxylin & Eosin

Cells were washed twice with sterile PBS and then fixed with an acetone-citrate solution (2:3 v/v; 0.038M citrate) for 30 seconds at room temperature. Slides were rinsed with sterile water, and fixed with Eosin Y for 5 minutes. Eosin was then aspirated, and slides washed twice with sterile water, incubated for 15 minutes with 1 mL of sterile water, and washed again twice. Slides were finally incubated with hematoxylin (a TRAP kit reagent, Sigma, cat# 285-2) for 5 minutes, incubated with sterile water for 3 minutes, dried, and mounted with an antifade reagent in glycerol buffer (SlowFade® Light) and sealed with acrylic glue.

### Hoechst-Phalloidin

Cells were washed three times with sterile PBS, and fixed with 4% formaldehyde in PBS for 10 minutes at room temperature. Cells were washed three more times with sterile PBS. Cells were then permeabilized with a 0.1% Triton® X-100 in PBS (0.22 μm filtered) for 2 minutes at room temperature and washed again with PBS. Finally, cells were stained with 1 mL of Hoechst (2 μg/mL in PBS) supplemented with 25 μL of Phalloidin stock solution. Slides were incubated in the dark at room temperature for 30 minutes, washed, gently rocked twice with sterile PBS, cleared, mounted with an antifade reagent in glycerol buffer (SlowFade® Light) and sealed with acrylic glue. Phalloidin stock solution was prepared by dissolving the lyophilized solid in 1.5 mL MeOH (200 units/mL≅6.6 μM). Hoechst stock solution was prepared by dissolving the solid in sterile water (1 mg/mL).

### Results

Osteoclasts were differentiated from BMNC cells as described in the Materials & Methods. In brief, bone marrow mononuclear cells (BMNC) were resuspended in either mesenchymal stem cell bulletkit medium (MSCGM) (Group B) or filtered α-MEM containing 5% fetal bovine serum (FBS) and granulocyte macrophage colony stimulating factor (GM-CSF) (Group C) and incubated overnight. Another group of cells received no overnight incubation (Group A). Cells were then cultured on a methylcellulose-based medium (MethoCult) to elicit their differentiation to mature colony-forming units of granulocytes and macrophages (CFU-GM). After 10 and 25 days of incubation, cells were counted and imaged using an inverted microscope. After 26 days of incubation, cells were washed with Iscove's modified dulbecco's medium (IMDM) and centrifuged, and cell viability was analyzed using trypan blue staining. Next, cells were seeded on plates or slides with IMDM containing 10% FBS, RANKL, and M-CSF to elicit osteoclast formation. Resorption of cells was evaluated using the OAAS plate system, and cells were stained for TRAP, H&E, and Hoechst-Phalloidin.

The protocol in which BMNC were not subjected to overnight pretreatment (Group A) provided the most robust results, with 95-100% of the cell colony demonstrating TRAP staining and histological markers of osteoclasts including multinuclearity. In addition, the protocol in which BMNC were subjected to overnight treatment with (Group C) or without (Group B) GM-CSF also provided high percentages of osteoclasts (85% and 90%, respectively).

### Example 2

### Osteoclast Functional Assay

Osteoclasts resulting from one of the differentiation protocols described above (Groups A-C) were evaluated for resorption activity as a measure of osteoclast function.

### Materials & Methods

Colony cells were cultured on an Osteoclast Activity Assay Substrate (OAAS™) 24 well plate (Osteogenic Core Technologies (OCT) Inc., Choongnam, Korea) with osteoclast medium containing dexamethasone (10⁻⁷ M) according to the osteoclast formation protocol. OAAS™ is a culture plate whose well bottoms are coated with phosphate, which acts as a substrate for the osteoclast. Cells are cultured for 2-3 days. The resorption area can be observed with the naked eye or using light microscopy.

### Results

Osteoclasts were differentiated from BMNC cells as described in the Materials & Methods above.

BMNC that received no pretreatment (Group A) produced the greatest percentage of mature, functional osteoclasts, as evidenced by an osteoclast activity assay. BMNC pretreated overnight in medium with (Group C) or without (Group B) GM-CSF also produced a high percentage of functional osteoclasts

In summary, BMNC that received no pretreatment (Group A) produced the greatest percentage of mature, functional osteoclasts, as evidenced by a) cell morphology revealed by H&E and Hoechst-Phalloidin stain, b) TRAP staining, and c) osteoclast activity assay. While overnight pretreatment of BMNC in medium with (Group C) or without (Group B) GM-CSF produced a high percentage of osteoclasts, the optimal protocol did not require overnight pretreatment of BMNC.

Advantages to this protocol include, inter alia, its ease of use, avoidance of overnight incubation, avoidance of a requirement to separate non-adherent from adherent BMNC before culturing, and use of centrifugation rather than manual isolation of CFU-GM. What is claimed is:

1. A method of producing a cell population enriched for osteoclasts, the method comprising the steps of:
(a) culturing bone marrow mononuclear cells (BMNC) in a medium supplemented with growth factors, cytokines, or a combination thereof, for at least 7 days to obtain colony-forming units of granulocytes and macrophages (CFU-GM);
(b) isolating said CFU-GM by centrifugation; and
(c) culturing said CFU-GM isolated in (b) for a period of time and under conditions sufficient for said CFU-GM to differentiate to osteoclasts.

2. The method of claim 1, wherein said BMNC are cultured in said medium for a period of time of approximately 7-35 days.

3. The method of claim 1, wherein said BMNC are cultured in said medium at a concentration of 75,000-800,000 cells/ml.

4. The method of claim 1, further comprising the step of incubating BMNC in non-viscous medium for 8-24 hours prior to step (a).

5. The method of claim 4, wherein said medium comprises granulocyte macrophage colony stimulating factor (GM-CSF).

6. A population of cells enriched for osteoclasts produced by the process of claim I.
